(19) United States
(12) Patent Application Publication
(10) Pub. No.: US 2021/0299378 A1
(43) Pub. Date: Sep. 30, 2021

(54) EMERGENCY VENTILATOR

(71) Applicant: Air Boost, LLC, Austin, TX (US)

(72) Inventors: Marc Christenson, Austin, TX (US); Austin Christensen, Cedar Park, TX (US); Jason Dearden, Austin, TX (US); Isaac Jones, Round Rock, TX (US); Jarod Horn, Austin, TX (US); Savan Patel, Austin, TX (US); Michael Morgan, Austin, TX (US); Brett Jacobson, Austin, TX (US); Alex Avila, Austin, TX (US); Vallan Sherrod, Austin, TX (US); Russell Aldridge, Austin, TX (US); Jacob Robinson, Round Rock, TX (US)

(73) Assignee: Air Boost, LLC, Austin, TX (US)

(21) Appl. No.: 17/219,480

(22) Filed: Mar. 31, 2021

Related U.S. Application Data
(60) Provisional application No. 63/002,703, filed on Mar. 31, 2020.

(51) Int. Cl. A61M 16/00; A61M 16/20

(52) U.S. Cl. CPC A61M 16/024; A61M 16/0078; A61M 16/20; A61M 2205/505; A61M 2205/3331; A61M 2230/42; A61M 2209/084; A61M 16/0051

(57) ABSTRACT

This disclosure describes systems, methods, and devices related to emergency ventilators. An emergency ventilator may determine one or more adjustable parameters associated with controlling a ventilator device to supply air to a user, wherein the one or more adjustable parameters are determined based at least in part on breathing thresholds associated with the user. The emergency ventilator may evaluate the adjustable parameters to generate a control signal. The emergency ventilator may cause one or more paddles to articulate based at least in part on the control signal, wherein the one or more paddles squeeze a bag valve mask (BVM) attached to the ventilator device. The emergency ventilator may generate one or more outputs associated with a condition of the ventilator device. The emergency ventilator may display, on a display device, the one or more outputs.

### CROSS-REFERENCE TO RELATED APPLICATION

This application claims the benefit of U.S. Provisional Application No. 62/002,703, filed Mar. 31, 2020, the disclosure of which is incorporated herein by reference as if set forth in full.

### TECHNICAL FIELD

This disclosure generally relates to systems, methods, and devices for medical devices and, more particularly, to an emergency ventilator.

### BACKGROUND

A ventilator is typically used as a form of therapy to treat patients with respiratory difficulties. Ventilation delivers breathable air to a patient at a predetermined rate. This creates an interactive system between the patient and the ventilator to provide adequate ventilation.

### BRIEF DESCRIPTION OF THE DRAWINGS

FIG. 1 depicts an illustrative schematic diagram for an emergency ventilator system, in accordance with one or more example embodiments of the present disclosure.

FIG. 2 depicts an illustrative schematic diagram of an emergency ventilator system, in accordance with one or more example embodiments of the present disclosure.

FIG. 3 depicts an illustrative schematic diagram of an illustrative 3-dimensional diagram of a chain drive assembly of the emergency ventilator system in an enclosed case, in accordance with one or more example embodiments of the present disclosure.

FIG. 4 depicts an illustrative schematic diagram of an illustrative 2-dimensional diagram of a chain drive assembly of the emergency ventilator system in an enclosed case, in accordance with one or more example embodiments of the present disclosure.

FIG. 5 depicts an illustrative schematic diagram of a display module of an emergency ventilator system, in accordance with one or more example embodiments of the present disclosure.

FIG. 6 depicts an illustrative plot for triggering a threshold in the emergency ventilator system, in accordance with one or more example embodiments of the present disclosure.

FIG. 7 depicts an illustrative schematic diagram of an example patient circuit, in accordance with one or more example embodiments of the present disclosure.

FIG. 8 depicts an illustrative schematic diagram of a display module of the emergency ventilator system, in accordance with one or more example embodiments of the present disclosure.

FIG. 9 depicts a flow diagram of an illustrative process for an emergency ventilator system, in accordance with one or more example embodiments of the present disclosure.

FIG. 10 depicts a block diagram of an example machine upon which any of one or more techniques (e.g., methods) may be performed, in accordance with one or more example embodiments of the present disclosure.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in or substituted for, those of other embodiments. Embodiments outlined in the claims encompass all available equivalents of those claims.

Example embodiments of the present disclosure relate to systems, methods, and devices for an emergency ventilator.

In one or more embodiments, an emergency ventilator system may be intended to be a low-cost ventilator that becomes quickly available to help in the ventilator shortage caused by COVID-19. The emergency ventilator addresses one or more issues presented by earlier ventilator models.

In one or more embodiments, an emergency ventilator system may be designed to be able to survive and operate in harsh environments, and survive rough handling in shipping and hospital settings. For example, the emergency ventilator system may be encased in a rugged plastic case that is rated for harsh environments, splash-proof (while closed) and can be shipped in that case without extra boxing or padding, if needed. Previous ventilators had exposed gearing in the squeezing mechanism. In contrast, these are covered by a main plate (e.g., metal plate, plastic plate, or any other type of plate) in the emergency ventilator system. Further, the emergency ventilator system may include adjustable mountings that could accommodate a larger range of resuscitator bags.

In one or more embodiments, the emergency ventilator system may be housed in a pelican case to provide flexibility in various environments. In that case, the electronics of the emergency ventilator system may be attached below the main plate, such that motion parts are enclosed within the pelican case except for the paddles. In some embodiments, an attachment may be added to the pelican case to allow an intravenous (IV) to be added as needed. Further, a mechanism may be added to the pelican case to allow the case to be secured in various orientations. For example, the pelican case may be attached to a pole using the mechanism.

In one or more embodiments, an emergency ventilator system may be a compact, fully transportable emergency ventilator that provides air to patients through the mechanism of having two controllable paddles squeeze a supported bag valve mask (BVM). The emergency ventilator system may be used by Qualified medical personnel who provide constant monitoring of a patient on ventilator support.

In one or more embodiments, an emergency ventilator system may facilitate one or more modes of operation. For example, the one or more modes of operation may include an OFF mode, an assist control mode, and/or a volume control mode. In the OFF mode, the emergency ventilator system may not actively be transferring air to the patients or assisting their breathing in any way.

In the assist mode, the emergency ventilator system may provide a volume pulse only when the system detects patient breathing. In the volume control mode, the emergency ventilator system may provide a constant volume pulse based on the tidal volume setting at a set respiratory rate. Further, there is a minimum respiratory rate and I:E ratio that the patient may be needed to meet otherwise the system will still ventilate and indicate an alarm.

In one or more embodiments, an emergency ventilator system may facilitate one or more functions. For example, the emergency ventilator system may facilitate a mechanical positive end-expiratory pressure (PEEP) valve setting. The mechanical PEEP valve setting may allow for continuous pressure to be set via a mechanical adjustment on an artificial manual breathing unit (AMBU) valve body. The emergency ventilator system may also facilitate setting the inspirations to expiration ratio associated with a respiratory rate. In one or more embodiments, an emergency ventilator system may facilitate one or more safety features such as the utilization of an overpressure valve used on the AMBU valve body.

The above descriptions are for purposes of illustration and are not meant to be limiting. Numerous other examples, configurations, processes, etc., may exist, some of which are described in detail below. Example embodiments will now be described with reference to the accompanying figures.

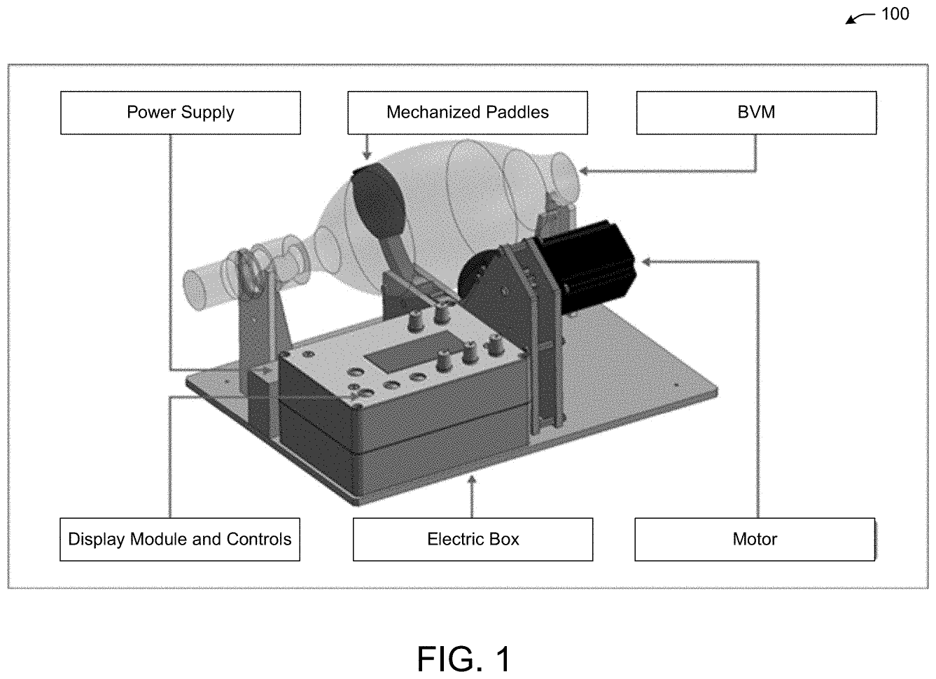

FIG. 1 depicts an illustrative schematic diagram for an emergency ventilator system 100, in accordance with one or more example embodiments of the present disclosure.

Referring to FIG. 1, there is shown an emergency ventilator system. The emergency ventilator system may be a compact, fully transportable ventilator that provides air to patients.

In one or more embodiments, the emergency ventilator system may be configured as an open frame design, where the electronics are located in a separate enclosure, such that the components of the emergency ventilator system are all attached to a main plate. The emergency ventilator may be equipped with a set of controllable mechanical paddles that are controlled by a ventilator controller device to squeeze a supported BVM and delivers the desired air volume at a given rate. The ventilator may be equipped with adjustable settings, modes, alarms, and options.

In one or more embodiments, an emergency ventilator system may provide automatic ventilation via squeezing AMBU/resuscitator bag. A resuscitator may be a device using positive pressure to inflate the lungs of a person who is not breathing or having difficulty breathing, in order to keep them oxygenated and alive.

In one or more embodiments, and in order to provide automatic ventilation, an emergency ventilator system may utilize a stepper motor, chain and sprocket drive train, and aluminum arms with plastic paddles that move in and out to squeeze the AMBU bag. The ventilation amount is controlled by a microcontroller device (e.g., Arduino-based, any other type of microcontroller device, a microprocessor, etc.), based on pressure and flow sensor readings, plus arm angle-to-tidal volume calibrations obtained experimentally.

In one or more embodiments, an emergency ventilator system may provide a durable solution that can survive shock and rough handling. The emergency ventilator system may be made from rugged, industrial components and designed to last for many cycles. It may also use advanced pressure and flow sensors to deliver accurate volumes and adapt to a patient who is beginning to breathe again on their own or is not fully sedated. This may an important aspect, as all recovering patients will need to be weaned off the ventilator.

In one or more embodiments, an emergency ventilator system may have been tested according to the standards based on the association for the advancement of medical instrumentation (AAMI). For example, AAMI CR501:2020 includes standards and testing procedures from ISO 80601-2-80, ISO 80601-2-12, ISO 80601-2-12, and IEC 60601-1, parts 1-8. Further, listed parameters and features of the device were tested according to these standards, and documentation of testing was submitted to the FDA for review. After a review of test results and documentation, the emergency ventilator system received emergency authorization for use on adult patients during the COVID-19 pandemic.

In one or more embodiments, an emergency ventilator system may facilitate motor angle to tidal volume calibration including PEEP pressure compensation. PEEP pressure is the last pressure measurement at the end of an expiratory phase before the beginning of the next inspiratory phase.

Every ventilation cycle the ventilator may deliver a volume of air to the patient based on the operator-set tidal volume (TV). This volume of air may be delivered by compressing the BVM some amount using articulating arms connected to a motor through a gear train. For some angle of motor turning, the BVM will be compressed a certain amount. Compressing the BVM a certain amount may push a certain volume of air through to the patient airway circuit. This volume of air may be constant for a given BVM compression assuming the patient airway pressure is constant. However, the patient airway pressure may be adjusted using the PEEP valve in the patient airway circuit. Therefore the tidal volume delivered may depend on two variables: motor angle and PEEP pressure. Using medical-grade scientific measurement tools, calibrations of Tidal Volume Delivered may be determined as a function of motor angle and PEEP, which may be used for accuracy of delivered air to a patient's lungs across many different settings (breaths per minute, max pressure, etc.).

In one or more embodiments, the emergency ventilator system may be configured to provide a plurality of controllable parameters. When these parameters are set by an operator, the emergency ventilator system may generate a signal (e.g., a control signal) that would be used to cause an action on or more parts of the emergency ventilator system. For example, based on the parameters, the emergency ventilator system may control a motor to move or articulate paddles that compress a bag valve mask (BVM) in order to supply air to a patient. Some of the parameters affect the amount of air being produced, etc. For example, some of the controllable parameters may include:

1) BPM (breaths per minute): between 8-40 BPM.

2) Tidal Volume (air volume pushed into the lung): between 200-800 mL based on patient weight.

3) I/E Ratio (inspiratory/expiration time ratio): recommended to start ˜1:2; adjustable between 1:1-1:4. Another controllable parameter: i time, which is another form of the I/E ratio (setting one sets the other for a given respiratory rate (BPM)). The limits of the i time are 0.3-3.75 seconds.

4) Threshold: when a patient tries to inspire they can cause a dip on the order of 1-5 cm H2O. This dip in pressure will trigger the next ventilation cycle when in assist control mode.

5) PEEP: 5-20 cmH2O, though many patients will need 10-15 cmH20. This value is set by the mechanical PEEP valve, not through the liquid-crystal display (LCD) interface.

In one or more embodiments, after an operator enters some of the parameters, they may be outputted to a display section of the emergency ventilator system. These parameters may be adjusted any time. It should be understood that the controllable parameters are for illustrative purposes and not intended to be limiting. Other controllable parameters may be included in the display module of the emergency ventilator system.

More examples of the controllable parameters are shown. For example, there is shown a clear alarm button. The clear alarm button on the display module allows the individual monitoring of the patient to clear any alarms that are actively sounding.

In one or more embodiments, the emergency ventilator system may be equipped with one or more safety features. For example, the safety features may include a plurality of alarms that may be displayed on the emergency ventilator system. Some of these alarms may include:

1) Pressure alarm, which is an alarm that will sound as a result of an unexpected and/or unacceptable respiratory pressure (high-pressure violations will trigger the alarm). This pressure limit is set at 40 cmH2O and cannot be changed. Some example of potential causes of a pressure alarm can be—but are not limited to—the following:

a. Water in the ventilator circuit.

b. Increased and/or thicker mucus or secretions blocking the airway.

c. Coughing.

d. Gagging.

e. Equipment leak or disconnect.

f. Tubing kinks.

g. The pressure sensor is not reading anything.

h. The pressure sensor is unexpectedly constant.

2) Faulty equipment alarm: an alarm will sound as a result of a perceived faulty equipment failure. Potential causes of a pressure alarm can be—but are not limited to—the following:

a. Failure of paddles reaching their “home”, or retracted positions.

b. Over temperature of the motor control circuitry.

c. Open circuit within the motor.

d. Over current within the motor circuit.

3) Start-Up Alarm: An alarm will sound upon turning on the emergency respirator system. This is to ensure that the device cannot get accidentally unplugged and then plugged back in without alerting staff. The ventilator will return to standby mode if it loses power. The proper mode for the patient will then need to be reset by the operator. Potential causes of a pressure alarm can be—but are not limited to—the following:

a. The cord is unplugged and then plugged back into the wall.

b. A power outage (a UPS battery backup option is available upon request).

c. Brownout.

d. Software malfunction.

4) Resetting a tripped alarm: to reset any tripped alarm press the Clear Alarm button on the display module. Resetting an alarm will clear the error from the screen and silence the alarm until another alarm is generated.

It should be noted that in the event of a tripped alarm, the display screen will alternate between the pressure display view (where it shows numerical values) and the alarm display view where it shows what problem/error is occurring.

In one or more embodiments, an operator may press the clear alarm button to view the most recent alarm. The operator can also go to the setting menu to view the past few alarms and see when they occurred. There is an asterisk that appears in the corner of the screen to notify the operator there is an alarm. This shows when an alarm is preset, even if the system has been set to silence alarms for a period of time. The asterisk may disappear once the operator pushes the reset alarm.

The Clear alarm button on the display module allows the operator monitoring the patient to clear any alarms that are actively sounding. Pushing the clear alarm button will cause the alarm to stop sounding and will then cause the alarm text to appear on the screen. Holding the clear alarm button down for 2 seconds will cause the alarm to be silenced, or to not sound, for 2 minutes. During that 2 minutes, an operator may press and hold the clear alarm button for 2 seconds again to re-enable the alarm sound.

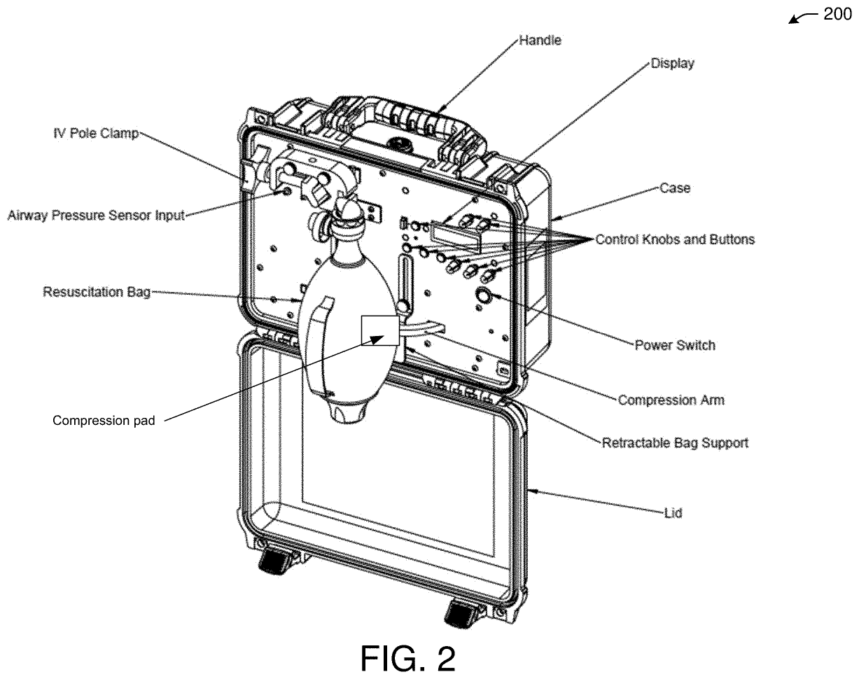

FIG. 2 depicts an illustrative schematic diagram 200 for an emergency ventilator system, in accordance with one or more example embodiments of the present disclosure.

In one or more embodiments, an emergency ventilator system may be encased in a rugged plastic case with a carrying handle, and also has a mounting clasp to affix the device to an intravenous (IV) pole or hospital gurney pole. Current ventilators sit flat on a tabletop surface, and cannot withstand shock or vibration from moving on a gurney or in an ambulance.

The enclosed emergency ventilator system 200 may comprise a case, a handle for the case, a lid to cover the emergency ventilator system, an IV pole clamp, an airway pressure sensor input, a resuscitation bag, a retractable bag support, one or more compression arms (only one is visible in FIG. 2A), a power switch, one or more control knobs and buttons, and a display.

In one or more embodiments, the emergency ventilator system may be constructed to house a variety of BVM resuscitators.

In one or more embodiments, a list of BVMs that may be supported may include parts listed in Table 1, however, it should be understood this list is only for illustrative purposes and other BVMs may be used.

TABLE 1
Various models of BMVs that may be used.

| Manufacturer | Part Number | Description |
|---|---|---|
| AMBU USA | 523211000 + | Adult Bag Reservoir BVM with Pop-off |
| | 199002020 | Valve, Medium Mask, Manometer Port |
| | | with optional PEEP valve |
| AMBU USA | 524611001 | Adult Tube Reservoir BVM with Pop-off |
| | | Valve, Medium Mask, Manometer Port |
| | | and included PEEP Valve |

It should be noted that the system may not be supplied with AMBU bags. These—and any connectors/hoses to connect the AMBU bag to the patient's face mask—will need to be provided by the hospital/doctor system administrators. Face masks may also not included with the system. It should also be noted that a patient circuit may not be included with the emergency ventilator system.

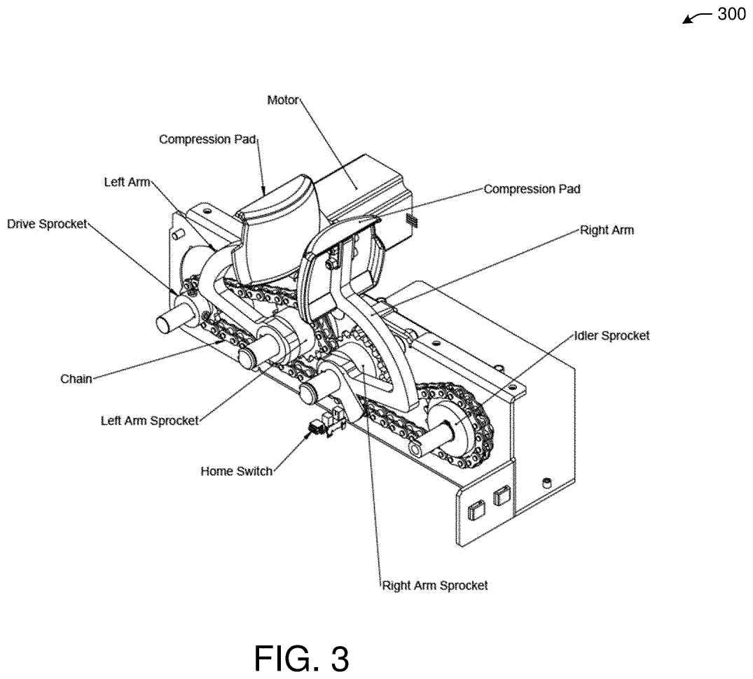

FIG. 3 depicts an illustrative schematic diagram of an illustrative 3-dimensional diagram 300 of a chain drive assembly of the emergency ventilator system in an enclosed case (e.g., a pelican case as shown in FIG. 2), in accordance with one or more example embodiments of the present disclosure.

In one or more embodiments, the chain drive assembly of the emergency ventilator system may comprise, a motor, two compression pads attached to two arms (left arm and right arm), the two arms may be attached to respective sprockets (right arm sprocket and left arm sprocket), a drive sprocket, an idler sprocket, and a home switch.

In one or more embodiments, the chain drive assembly of the emergency ventilator system may be attached below a main plate of a pelican case or any other type of enclosure, such that motion parts are enclosed within the pelican case except for the paddles. In some embodiments, an attachment may be added to the pelican case to allow IV to be added as needed. Further, a mechanism may be added to the pelican case to allow the case to be secured in various orientations. For example, the pelican case may be attached to a pole using the mechanism.

It is understood that the above descriptions are for purposes of illustration and are not meant to be limiting.

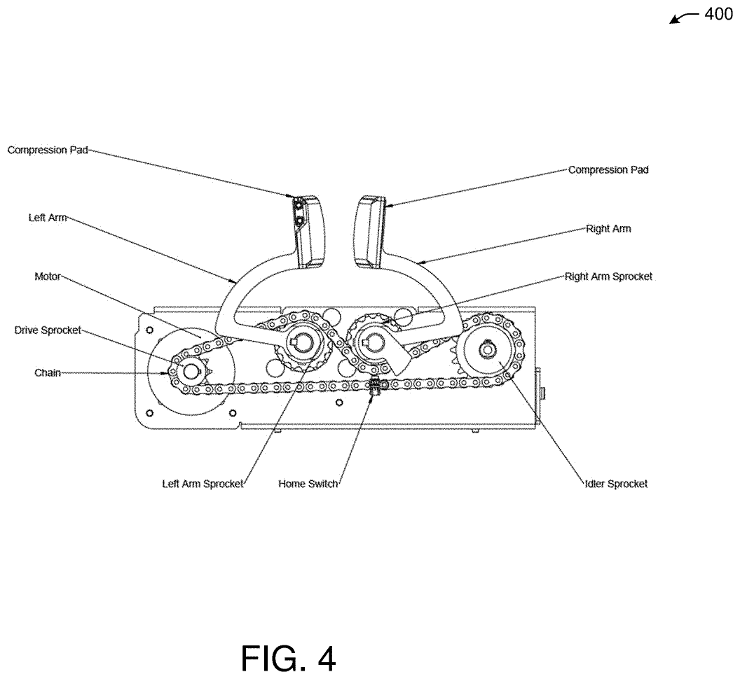

FIG. 4 depicts an illustrative schematic diagram of an illustrative 2-dimensional diagram 400 of a chain drive assembly of the emergency ventilator system in an enclosed case (e.g., a pelican case as shown in FIG. 2), in accordance with one or more example embodiments of the present disclosure.

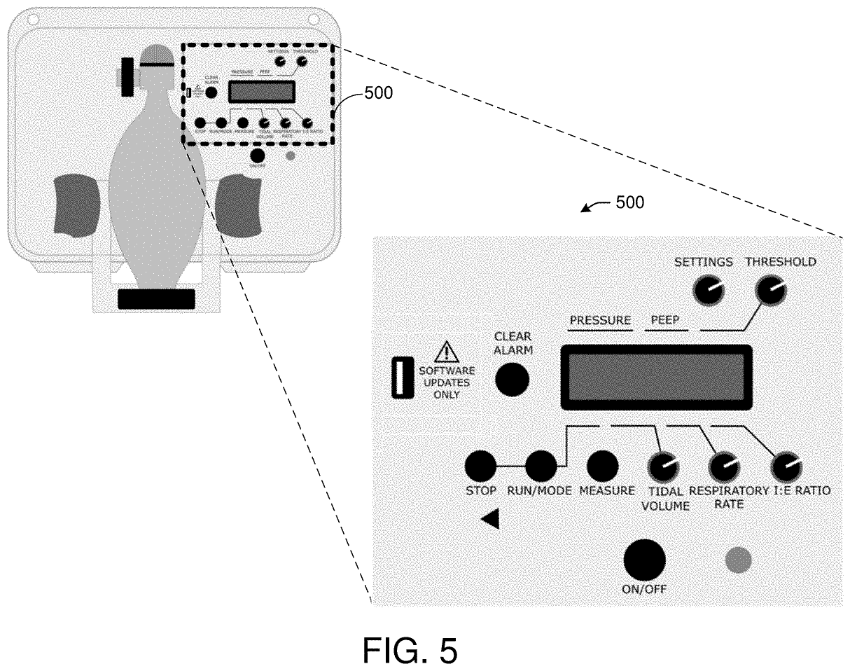

FIG. 5 depicts an illustrative schematic diagram of a display module 500 of an emergency ventilator system, in accordance with one or more example embodiments of the present disclosure.

Referring to FIG. 5, there is shown a display module 500 (e.g., the display and the one or more control knobs and buttons of FIG. 2). On the display module 500, there may be a display screen, a software updates port (e.g., a USB port), a clear alarm button, a stop button, a run/mode button, a measure button, a tidal volume dial (control know), a respiratory rate dial, an I:E ratio dial, a settings dial, and a threshold dial.

In one or more embodiments, to activate the settings menu, the settings dial may be used. Once the settings dial is pushed down, the settings menu becomes visible on the screen. To navigate through the settings menu, the settings dial may be turned clockwise and clicked when the desired setting title is selected.

In one or more embodiments, the threshold is the decrease in airway pressure that will trigger the next breath during assist control (AC). This pressure is created by the patient's attempts to inhale. During AC mode, the volume cycled ventilation may be triggered by the patient's inspiratory efforts. If the patient does not attempt to inhale, inspiration will automatically be delivered at regular intervals. The volume is determined by the operator using the dials and controls. The demand pressure is relative to the PEEP pressure and the threshold pressure. The Threshold dial is adjustable by increments of 1 cmH2O with a threshold limit between 1-5 cmH2O.

Volume control mode may be a mode where the emergency ventilator system runs similarly to running in the AC mode, except that it does not try to sense the inhale from the patient. Volume control delivers a specified volume at a set respiratory rate (e.g., 8-40 breaths per minute). This mode does not wait for or accommodate spontaneous inspiratory effort by the patient. Patients are most often sedated during this mode. It should be noted that if the patient tries to take a breath in volume control mode the system will allow—but not assist—the patient's “off-cycle” breath and will not change the breathing cycle rate.

The pressure indication may be a unit of centimeter (cm) water (H2O), the PEEP indication may be a unit of cmH2O. The PEEP display on the LCD is showing the measured PEEP value so an operator can compare it to the value set on the mechanical PEEP valve. The settings on the mechanical PEEP valves may not be fully reliable so this measurement helps the operator dial in the exact PEEP value needed.

The PEEP indicator shows the PEEP levels and is measured once a cycle. Turning the PEEP valve up will increase the PEEP read-out on the display screen while turning it down will decrease the PEEP read-out on the display screen.

The clear alarm button on the display module allows the individual monitoring the patient to clear any alarms that are actively sounding.

The stop is a mode in which the emergency ventilator system is not actively transferring air to the patient or assisting their breathing in any way. After pressing the stop button the screen will prompt the operator (e.g., a doctor, a nurse, a clinician, a technician, etc.) to press the stop button again by displaying “PRESS STOP AGAIN TO STOP SYSTEM.”

The Run/Mode button allows the operator to run the unit in controlled mechanical ventilation (CMV) mode. In CMV mode the ventilator may deliver a specified volume at a set respiratory rate (e.g., 8-40 breath per minute). This mode may not wait for or accommodate spontaneous inspiratory effort by the patient. In some scenarios, patients may be sedated during the use of this mode.

The measure button measures the peak and plateau pressures on the next ventilation cycle and then reports them on the display screen. Peak pressure is the highest pressure recorded during the inspiratory phase. Plateau pressure is the pressure measured between an inspiratory and expiratory phase after a 0.5 second pause.

Tidal volume is the volume of air delivered to the patient's lungs with each breath by the emergency use resuscitator and is measured in milliliter (mL). Using this tidal volume dial, the tidal volume of mechanical breaths can be adjusted in 25 mL increments. Tidal volume is to be set by a clinician based on the individual patient. 6-8 ml/kg ideal patient body weight may be considered the starting point for setting the tidal volume, but it must be adjusted for a patient's individual circumstances (such as reduced lung capacity, etc.). The tidal volume dial is adjustable by increments of mL (e.g., increments of 25 mL, 50 mL, etc.).

The Respiratory Rate dial may allow for a set number of breaths per minute (BPM) to be input into the emergency use resuscitator. For example, if the Respiratory Rate is set at 10, the resuscitator will deliver 10 breaths per minute, or a breath every 6 seconds. The Respiratory Rate dial is adjustable by increments of 1 breath per minute. It should be noted that in Assist Control, a patient can have a natural respiratory rate faster than what the machine is set to and the system will assist their breathing. The patient cannot breathe slower than the setting as doing so will trigger an automatic breath and sound an alarm.

Inspiratory:Expiratory ratio refers to the ratio of inspiratory time to expiratory time. For instance, in normal spontaneous breathing, the expiratory time tends to be about twice as long as the inspiratory time. This gives an I:E ratio of 1:2. This dial allows for a set I:E ratio to be input into the emergency use resuscitator. The I:E ratio dial is currently adjustable by increments of 1 (range is 1:1 to 1:4). Further, an operator may choose a specific “i-time” (e.g., in seconds) that the inspiratory phase can last. The i-time may be limited to times that may keep the I:E ratio between 1:1 and 1:4. For example, pushing down on the I:E ratio dial will take the operator to the i-time page, and turning the I:E ratio dial will let the operator update the i-time and the respiratory rate. Since the i-time, I/E ratio, and BPM represent an over-constrained problem (the operator may only need two of those values to define the ventilation rate), when changing one value, one of the other two values will also change in order to match. For example, for a given BPM, changing the I/E ratio will force the i-time to update to match the set BPM and I/E ratio.

The ON/OFF button turns the system ON or OFF. The OFF mode is a mode in which the emergency ventilator system is not actively transferring air to the patients or assisting their breathing in any way.

It is understood that the above descriptions are for purposes of illustration and are not meant to be limiting.

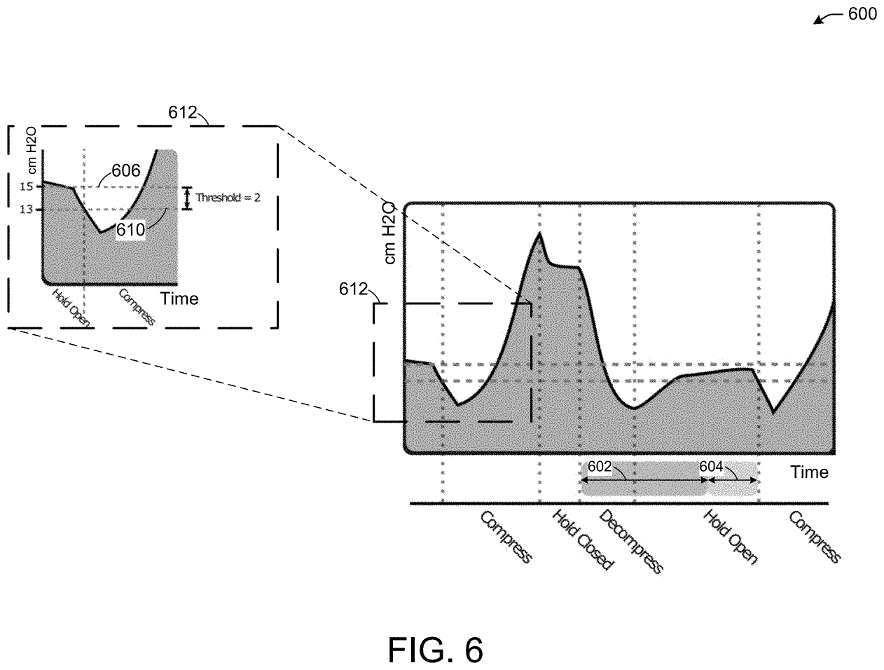

FIG. 6 depicts an illustrative plot for triggering a threshold in the emergency ventilator system, in accordance with one or more example embodiments of the present disclosure.

Referring to FIG. 6, there is shown plot having the X-axis in time and the Y-axis in cmH2). An exploded view of a portion 612 is shown where a threshold value is set to be 2 cmH2O.

In one or more embodiments, an emergency ventilator system may comprise an adjustable threshold delay time (in percentage) during the assist control mode based on the patient's needs. The operator can set a period after inhalation where the patient cannot trigger another breath for a certain period of time and the operator can adjust that threshold (delay time) in order to avoid having breaths triggered back to back. The threshold is a percentage of a time between finishing an inhalation and when the next inhalation would occur. There is a minimum repertory rate, when in assist control mode, that the system has to run at, even if the patient does not take a breath. For example, assuming there is a one second gap in between breaths, then assuming the percentage is 60%, then the system would wait for 0.6 seconds before it starts looking for another breath trigger. Looking at time period 602, it represents the phase where the emergency ventilator would “ignore crossing threshold,” and the time period 604 represents the phase where the emergency ventilator would “accept crossing threshold.” If the system measures a pressure below 13, during the ignore crossing threshold phase, the system would not register this crossing of the threshold as a trigger for another breath. The threshold is a pressure difference between the PEEP value and the measured pressure. However, the threshold delay time percent is a percentage. The threshold indicates the pressure at which to trigger another breath.

In the example of FIG. 6, the PEEP is measured at 15 cmH2O with a threshold set to 2. If the emergency ventilator system measures a pressure that is below 13 (PEEP minus Threshold) during the ignore crossing threshold phase, compression will not start. If the emergency ventilator system measures a pressure drop below 13 (PEEP minus Threshold) during the accept crossing threshold phase, the emergency ventilator system will trigger a new inspiration and compress.

It is understood that the above descriptions are for purposes of illustration and are not meant to be limiting.

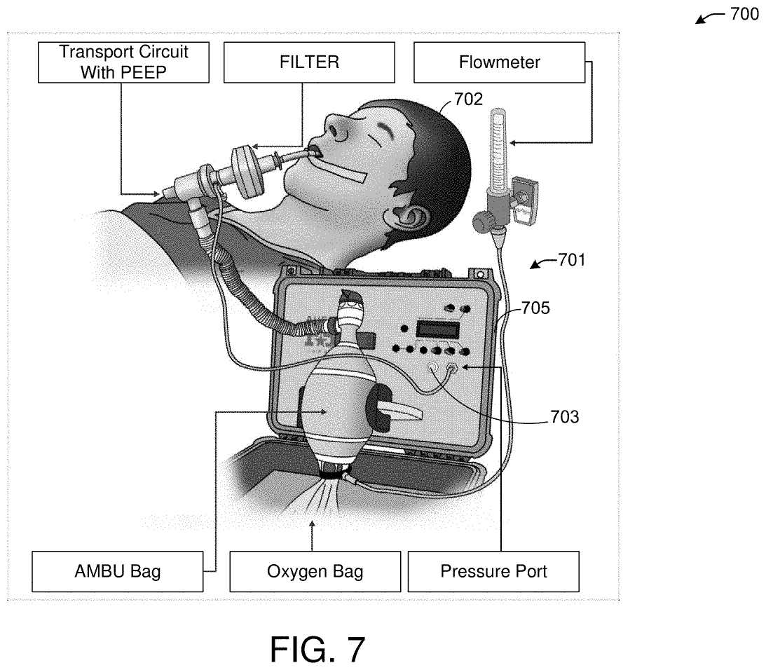

FIG. 7 depicts an illustrative schematic diagram 700 of an example patient circuit, in accordance with one or more example embodiments of the present disclosure.

Referring to FIG. 7 there is shown an example patient circuit that comprises an emergency ventilator system 701 that is attached to a patient 702.

The patient circuit is the artificial conduit that relays gases between a ventilator/resuscitator (e.g., the emergency ventilator system 701) and the patient 702.

The example patient circuit may comprise a transport circuit with a PEEP valve (e.g., a Westmed transport circuit or any other transport circuit), a filter (e.g., ISO-Gard 28022 HEPA filter, or any other types filters based on the implementation), a flowmeter, a BVM (e.g., an AMBU bag, or other types of bags), a pressure port, and an oxygen bag.

During a ventilation cycle, the ventilator may deliver a volume of air to the patient 702 based on the operator-set tidal volume. This volume of air may be delivered by compressing the BVM some amount using articulating arms connected to a motor through a gear train.

The pressure port connection is located next to the power switch 703 in the pelican case 705. This connection point is where an operator (e.g., a doctor, a nurse, a clinician, etc.) may affix the pressure port which is used to monitor the patient's breathing.

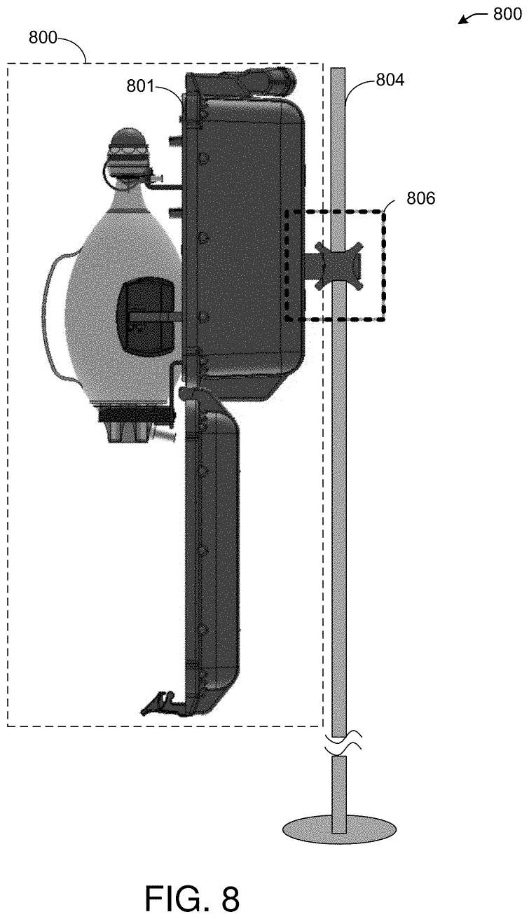

FIG. 8 depicts an illustrative schematic diagram of a display module of the emergency ventilator system 800, in accordance with one or more example embodiments of the present disclosure.

In one or more embodiments, the BVM may be held securely in place by one or more Velcro straps. For example, a Velcro strap will be affixed to each end of the BVM (the Velcro straps may not be placed on the bag itself, but placed on the valves sticking out of each end).

In one or more embodiments, the emergency ventilator system 800 may be housed in a pelican case 801 to provide flexibility in various environments. In that case, the electronics of the emergency ventilator system 800 may be attached below a main plate, such that motion parts are enclosed within the pelican case 801 except for the paddles. In some embodiments, an attachment (not shown here) may be added to the pelican case to allow an intravenous (IV) to be added as needed. Further, a mechanism 806 may be added to the pelican case to allow the case to be secured in various orientations. For example, the pelican case 801 may be attached to a pole 804 using the mechanism 806.

In one or more embodiments, an emergency ventilator system may be capable of being mobile and portable.

In one or more embodiments, an emergency ventilator system may facilitate that the display screen comprises a large display visible from a distance that may span across one room. Further, the display screen may be vertical facing to facilitate increased visibility.

In one or more embodiments, one or more knobs/buttons may have detents and may facilitate click feedback.

In one or more embodiments, the alarms on the emergency ventilator system may comprise auditory and blinking of light to indicate that something needs attention. Further, such alarms may also be reset by an operator such as a nurse. The alarm may remain/persist for an extended time to get the attention of an operator such as a nurse. The operator can press and hold the reset alarm button until they hear a beep (e.g., hold for 2 seconds). After that, the system will not make an audible sound if alarms are raised during the next 2 minutes. It is understood that the above descriptions are for purposes of illustration and are not meant to be limiting.

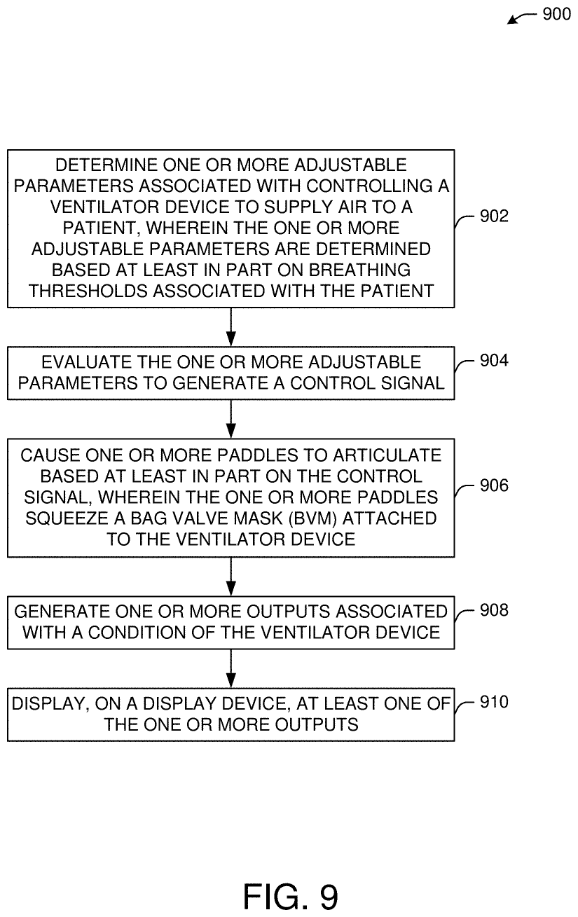

FIG. 9 and illustrates a flow diagram of illustrative process 900 for an illustrative emergency ventilator system, in accordance with one or more example embodiments of the present disclosure.

At block 902, an emergency ventilator system (e.g., the emergency ventilator system of FIGS. 1 and/or 2) may determine one or more adjustable parameters associated with controlling a ventilator device to supply air to a user, wherein the one or more adjustable parameters are determined based at least in part on breathing thresholds associated with the user. The one or more adjustable parameters may include at least one of a pressure parameter, a mechanical positive end-expiratory pressure (PEEP) parameter, a tidal volume, a threshold, a respiratory rate, a control mode, an inspiratory and expiratory ratio, or an additional time (e.g., +i-time). A control mode of the one or more adjustable parameters may comprise an OFF mode, an assist control mode, or a volume control mode. The emergency ventilator system may raise an alarm based on detecting a state of the ventilator device. The controller device may be further configured to determine a threshold delay percent that is set by an operator to determine a percentage of time at which a pressure drop below a threshold is ignored. The controller device is further configured to use a threshold pressure value for determining when a next ventilation cycle in an assist mode will be triggered.

At block 904, the emergency ventilator system may evaluate the adjustable parameters to generate a control signal. The control signal may cause a motor of the ventilator device to output power resulting in articulating one or more paddles. The alarm is at least one of a pressure alarm, a faulty equipment alarm, a start-up alarm, or a reset alarm. A pressure difference between a mechanical positive end-expiratory pressure (PEEP) pressure value and a measured pressure may be greater than a threshold pressure value, the controller device causes a compression based on the threshold delay percent. The ventilator system is enclosed in a pelican case, wherein the pelican case or any type of enclosure case, where the pelican case comprises an intravenous (IV) attachment or a pole attachment.

At block 906, the emergency ventilator system may cause one or more paddles to articulate based at least in part on the control signal, wherein the one or more paddles squeeze a bag valve mask (BVM) attached to the ventilator device. The BVM may be attached to a mechanical positive end-expiratory pressure (PEEP) valve.

At block 908, the emergency ventilator system may generate one or more outputs associated with a condition of the ventilator device.

At block 910, the emergency ventilator system may display, on a display device, at least one of the one or more outputs.

It is understood that the above descriptions are for purposes of illustration and are not meant to be limiting.

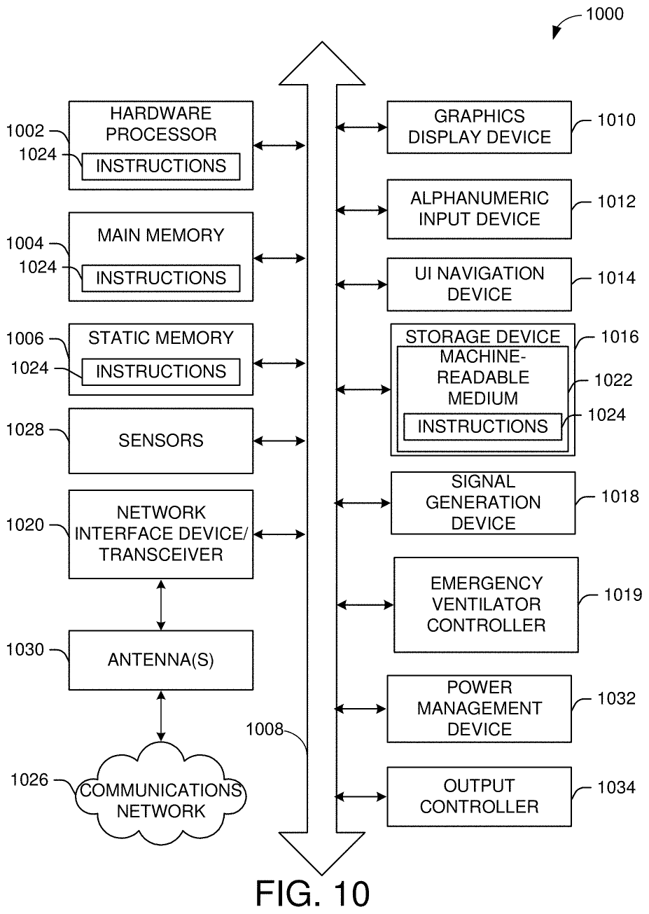

FIG. 10 illustrates a block diagram of an example of a machine 1000 or system upon which any one or more of the techniques (e.g., methodologies) discussed herein may be performed. In other embodiments, the machine 1000 may operate as a standalone device or may be connected (e.g., networked) to other machines. In an example, the machine 1000 may act as a peer machine in peer-to-peer (P2P) (or other distributed) network environments. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), or other computer cluster configurations.

Examples, as described herein, may include or may operate on logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In another example, the hardware may include configurable execution units (e.g., transistors, circuits, etc.) and a computer-readable medium containing instructions where the instructions configure the execution units to carry out a specific operation when in operation. The configuring may occur under the direction of the executions units or a loading mechanism. Accordingly, the execution units are communicatively coupled to the computer-readable medium when the device is operating. In this example, the execution units may be a member of more than one module. For example, under operation, the execution units may be configured by a first set of instructions to implement a first module at one point in time and reconfigured by a second set of instructions to implement a second module at a second point in time.

The machine (e.g., computer system) 1000 may include a hardware processor 1002 (e.g., a central processing unit (CPU), a microprocessor, a display LCD, one or more knobs, one or more buttons for a user interface (UI) control, a motor drive circuit, a pressure transducer, a speaker, a graphics processing unit (GPU), a hardware processor core, or any combination thereof), a main memory 1004 and a static memory 1006, some or all of which may communicate with each other via an interlink (e.g., bus) 1008. The machine 1000 may further include a power management device 1032, a graphics display device 1010, an alphanumeric input device 1012 (e.g., a keyboard), and a UI navigation device 1014 (e.g., a mouse, a button, a knob, a dial, etc.). For example, the graphics display device 1010, alphanumeric input device 1012, and UI navigation device 1014 may be a touch screen display. The machine 1000 may additionally include a storage device (i.e., drive unit) 1016, a signal generation device 1018 (e.g., a speaker), an emergency ventilator controller 1019, a network interface device/transceiver 1020 coupled to antenna(s) 1030, and one or more sensors 1028, such as a global positioning system (GPS) sensor, a compass, an accelerometer, or other sensors. The machine 1000 may include an output controller 1034, such as a serial (e.g., universal serial bus (USB), parallel, or other wired or wireless (e.g., infrared (IR), near field communication (NFC), etc.) connection to communicate with or control one or more peripheral devices (e.g., a printer, a card reader, etc.)). The storage device 1016 may include a machine-readable medium 1022 on which is stored one or more sets of data structures or instructions 1024 (e.g., software) embodying or utilized by any one or more of the techniques or functions described herein. The instructions 1024 may also reside, completely or at least partially, within the main memory 1004, within the static memory 1006, or within the hardware processor 1002 during execution thereof by the machine 1000. In an example, one or any combination of the hardware processor 1002, the main memory 1004, the static memory 1006, or the storage device 1016 may constitute machine-readable media.

The emergency ventilator controller 1019 may carry out or perform any of the operations and processes (e.g., process 900) described and shown above.

It is understood that the above are only a subset of what the emergency ventilator controller 1019 may be configured to perform and that other functions included throughout this disclosure may also be performed by the emergency ventilator controller 1019.

While the machine-readable medium 1022 is illustrated as a single medium, the term “machine-readable medium” may include a single medium or multiple media (e.g., a centralized or distributed database, and/or associated caches and servers) configured to store the one or more instructions 1024.

Various embodiments may be implemented fully or partially in software and/or firmware. This software and/or firmware may take the form of instructions contained in or on a non-transitory computer-readable storage medium. Those instructions may then be read and executed by one or more processors to enable the performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read-only memory (ROM); random access memory (RAM); magnetic disk storage media; optical storage media; a flash memory, etc.

The term “machine-readable medium” may include any medium that is capable of storing, encoding, or carrying instructions for execution by the machine 1000 and that cause the machine 1000 to perform any one or more of the techniques of the present disclosure, or that is capable of storing, encoding, or carrying data structures used by or associated with such instructions. Non-limiting machine-readable medium examples may include solid-state memories and optical and magnetic media. In an example, a massed machine-readable medium includes a machine-readable medium with a plurality of particles having resting mass. Specific examples of massed machine-readable media may include non-volatile memory, such as semiconductor memory devices (e.g., electrically programmable read-only memory (EPROM), or electrically erasable programmable read-only memory (EEPROM)) and flash memory devices; magnetic disks, such as internal hard disks and removable disks; magneto-optical disks; and CD-ROM and DVD-ROM disks.

The instructions 1024 may further be transmitted or received over a communications network 1026 using a transmission medium via the network interface device/transceiver 1020 utilizing any one of a number of transfer protocols (e.g., frame relay, internet protocol (IP), transmission control protocol (TCP), user datagram protocol (UDP), hypertext transfer protocol (HTTP), etc.). Example communications networks may include a local area network (LAN), a wide area network (WAN), a packet data network (e.g., the Internet), mobile telephone networks (e.g., cellular networks), plain old telephone (POTS) networks, wireless data networks (e.g., Institute of Electrical and Electronics Engineers (IEEE) 802.10 family of standards known as Wi-Fi®, IEEE 802.16 family of standards known as WiMax®), IEEE 802.15.4 family of standards, and peer-to-peer (P2P) networks, among others. In an example, the network interface device/transceiver 1020 may include one or more physical jacks (e.g., Ethernet, coaxial, or phone jacks) or one or more antennas to connect to the communications network 1026. In an example, the network interface device/transceiver 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1000 and includes digital or analog communications signals or other intangible media to facilitate communication of such software. The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

The word “exemplary” is used herein to mean “serving as an example, instance, or illustration.” Any embodiment described herein as “exemplary” is not necessarily to be construed as preferred or advantageous over other embodiments. The terms “computing device,” “user device,” “communication station,” “station,” “handheld device,” “mobile device,” “wireless device” and “user equipment” (UE) as used herein refers to a wireless communication device such as a cellular telephone, a smartphone, a tablet, a netbook, a wireless terminal, a laptop computer, a femtocell, a high data rate (HDR) subscriber station, an access point, a printer, a point of sale device, an access terminal, or other personal communication system (PCS) device. The device may be either mobile or stationary.

As used within this document, the term “communicate” is intended to include transmitting, or receiving, or both transmitting and receiving. This may be particularly useful in claims when describing the organization of data that is being transmitted by one device and received by another, but only the functionality of one of those devices is required to infringe the claim. Similarly, the bidirectional exchange of data between two devices (both devices transmit and receive during the exchange) may be described as “communicating,” when only the functionality of one of those devices is being claimed. The term “communicating” as used herein with respect to a wireless communication signal includes transmitting the wireless communication signal and/or receiving the wireless communication signal. For example, a wireless communication unit, which is capable of communicating a wireless communication signal, may include a wireless transmitter to transmit the wireless communication signal to at least one other wireless communication unit, and/or a wireless communication receiver to receive the wireless communication signal from at least one other wireless communication unit.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or in any other manner.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a controller device configured to execute computer-executable instructions to: determine one or more adjustable parameters associated with supplying air to a patient. The ventilator system also includes evaluate the one or more adjustable parameters to generate a control signal; and generate one or more outputs associated with the adjustable parameters. The ventilator system also includes one or more paddles, where the one or more paddles are positioned around a bag valve mask (BVM) attached to the ventilator system; a motor adapted to articulate the one or more paddles based at least in part on the control signal generated by the controller device, and a display device to display at least one of the one or more outputs generated by the controller device. Other embodiments of this aspect include corresponding computer systems, apparatus, and computer programs recorded on one or more computer storage devices, each configured to perform the actions of the methods.

Implementations may include one or more of the following features. The ventilator system where the one or more adjustable parameters include at least one of a pressure parameter, a mechanical positive end-expiratory pressure (PEEP) parameter, a tidal volume, a threshold, a respiratory rate, a control mode, an inspiratory and expiratory ratio, or an additional time. The controller device is further configured to determine a threshold delay percent that is set by an operator to determine a percentage of time at which a pressure drop below a threshold is ignored. The controller device is further configured to use a threshold pressure value for determining when a next ventilation cycle in an assist mode will be triggered. A pressure difference between a mechanical positive end-expiratory pressure (PEEP) pressure value and a measured pressure is greater than a threshold pressure value, the controller device causes a compression based on the threshold delay percent. The ventilator system is enclosed in a pelican case, where the pelican case. The pelican case includes an intravenous (IV) attachment or a pole attachment. Articulating the one or more paddles causes the one or more paddles to squeeze the BVM. The BVM is attached to a mechanical positive end-expiratory pressure (PEEP) valve. The controller device raises an alarm based on detecting a state of the ventilator system. The alarm is at least one of a pressure alarm, a faulty equipment alarm, a start-up alarm, or a reset alarm. Implementations of the described techniques may include hardware, a method or process, or computer software on a computer-accessible medium.

One general aspect includes a device having a processor configured to execute computer-executable instructions to: determine one or more adjustable parameters associated with controlling a ventilator device to supply air to a patient, where the one or more adjustable parameters are determined based at least in part on breathing thresholds associated with the patient; evaluate the one or more adjustable parameters to generate a control signal; cause one or more paddles to articulate based at least in part on the control signal, where the one or more paddles squeeze a bag valve mask (BVM) attached to the ventilator device; generate one or more outputs associated with a condition of the ventilator device; and display, on a display device, at least one of the one or more outputs. Other embodiments of this aspect include corresponding computer systems, apparatus, and computer programs recorded on one or more computer storage devices, each configured to perform the actions of the methods.

Implementations may include one or more of the following features. The device where the processor is further configured to determine a threshold delay percent that is set by an operator to determine a percentage of time at which a pressure drop below a threshold is ignored. The processor is further configured to use a threshold pressure value for determining when a next ventilation cycle in an assist mode will be triggered. A pressure difference between a mechanical positive end-expiratory pressure (PEEP) pressure value and a measured pressure is greater than a threshold pressure value, the controller device causes a compression based on the threshold delay percent. The ventilator device is enclosed in a pelican case. The pelican case includes an intravenous (IV) attachment or a pole attachment. Implementations of the described techniques may include hardware, a method or process, or computer software on a computer-accessible medium.

One general aspect includes a method. The method includes determining, by a processor of a ventilator controller device, one or more adjustable parameters associated with controlling a ventilator device to supply air to a user, where the one or more adjustable parameters are determined based at least in part on breathing thresholds associated with the user; evaluating the one or more adjustable parameters to generate a control signal; causing one or more paddles to articulate based at least in part on the control signal, where the one or more paddles squeeze a bag valve mask (BVM) attached to the ventilator device; generating one or more outputs associated with a condition of the ventilator device; and displaying, on a display device, at least one of the one or more outputs. Other embodiments of this aspect include corresponding computer systems, apparatus, and computer programs recorded on one or more computer storage devices, each configured to perform the actions of the methods.

Implementations may include one or more of the following features. The method further including setting a threshold delay percent by an operator to determine a percentage of time at which a pressure drop below a threshold is ignored. The method further including using a threshold pressure value for determining when the next ventilation cycle in an assist mode will be triggered. Implementations of the described techniques may include hardware, a method or process, or computer software on a computer-accessible medium.

Some embodiments may be used in conjunction with various devices and systems, for example, a personal computer (PC), a desktop computer, a mobile computer, a laptop computer, a notebook computer, a tablet computer, a server computer, a handheld computer, a handheld device, a personal digital assistant (PDA) device, a handheld PDA device, an on-board device, an off-board device, a hybrid device, a vehicular device, a non-vehicular device, a mobile or portable device, a consumer device, a non-mobile or non-portable device, a wireless communication station, a wireless communication device, a wireless access point (AP), a wired or wireless router, a wired or wireless modem, a video device, an audio device, an audio-video (A/V) device, a wired or wireless network, a wireless area network, a wireless video area network (WVAN), a local area network (LAN), a wireless LAN (WLAN), a personal area network (PAN), a wireless PAN (WPAN), and the like.

Some embodiments may be used in conjunction with one way and/or two-way radio communication systems, cellular radio-telephone communication systems, a mobile phone, a cellular telephone, a wireless telephone, a personal communication system (PCS) device, a PDA device which incorporates a wireless communication device, a mobile or portable global positioning system (GPS) device, a device which incorporates a GPS receiver or transceiver or chip, a device which incorporates an RFID element or chip, a multiple input multiple output (MIMO) transceiver or device, a single input multiple output (SIMO) transceiver or device, a multiple input single output (MISO) transceiver or device, a device having one or more internal antennas and/or external antennas, digital video broadcast (DVB) devices or systems, multi-standard radio devices or systems, a wired or wireless handheld device, e.g., a smartphone, a wireless application protocol (WAP) device, or the like.

Certain aspects of the disclosure are described above with reference to block and flow diagrams of systems, methods, apparatuses, and/or computer program products according to various implementations. It will be understood that one or more blocks of the block diagrams and flow diagrams, and combinations of blocks in the block diagrams and the flow diagrams, respectively, may be implemented by computer-executable program instructions. Likewise, some blocks of the block diagrams and flow diagrams may not necessarily need to be performed in the order presented, or may not necessarily need to be performed at all, according to some implementations.

These computer-executable program instructions may be loaded onto a special-purpose computer or other particular machine, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable storage media or memory that may direct a computer or other programmable data processing apparatus to function in a particular manner, such that the instructions stored in the computer-readable storage media produce an article of manufacture including instruction means that implement one or more functions specified in the flow diagram block or blocks. As an example, certain implementations may provide for a computer program product, comprising a computer-readable storage medium having a computer-readable program code or program instructions implemented therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language is not generally intended to imply that features, elements, and/or operations are in any way required for one or more implementations or that one or more implementations necessarily include logic for deciding, with or without user input or prompting, whether these features, elements, and/or operations are included or are to be performed in any particular implementation.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A ventilator system comprising:
a controller device configured to execute computer-executable instructions to:
determine one or more adjustable parameters associated with supplying air to a patient;
evaluate the one or more adjustable parameters to generate a control signal; and
generate one or more outputs associated with the adjustable parameters;
one or more paddles, wherein the one or more paddles are positioned around a bag valve mask (BVM) attached to the ventilator system;
a motor adapted to articulate the one or more paddles based at least in part on the control signal generated by the controller device; and
a display device to display at least one of the one or more outputs generated by the controller device.

2. The ventilator system of claim 1, wherein the one or more adjustable parameters include at least one of a pressure parameter, a mechanical positive end-expiratory pressure (PEEP) parameter, a tidal volume, a threshold, a respiratory rate, a control mode, an inspiratory and expiratory ratio, or an additional time.

3. The ventilator system of claim 1, wherein the controller device is further configured to determine a threshold delay percent that is set by an operator to determine a percentage of time at which a pressure drop below a threshold is ignored.

4. The ventilator system of claim 3, wherein the controller device is further configured to use a threshold pressure value for determining when a next ventilation cycle in an assist mode will be triggered.

5. The ventilator system of claim 3, wherein a pressure difference between a mechanical positive end-expiratory pressure (PEEP) pressure value and a measured pressure is greater than a threshold pressure value, the controller device causes a compression based on the threshold delay percent.

6. The ventilator system of claim 1, wherein the ventilator system is enclosed in a pelican case, wherein the pelican case.

7. The ventilator system of claim 6, wherein the pelican case comprises an intravenous (IV) attachment or a pole attachment.

8. The ventilator system of claim 1, wherein articulating the one or more paddles causes the one or more paddles to squeeze the BVM.

9. The ventilator system of claim 1, wherein the BVM is attached to a mechanical positive end-expiratory pressure (PEEP) valve.

10. The ventilator system of claim 1, wherein the controller device raises an alarm based on detecting a state of the ventilator system.

11. The ventilator system of claim 10, wherein the alarm is at least one of a pressure alarm, a faulty equipment alarm, a start-up alarm, or a reset alarm.

12. A device, comprising:
a processor configured to execute computer-executable instructions to:
determine one or more adjustable parameters associated with controlling a ventilator device to supply air to a patient, wherein the one or more adjustable parameters are determined based at least in part on breathing thresholds associated with the patient;
evaluate the one or more adjustable parameters to generate a control signal;
cause one or more paddles to articulate based at least in part on the control signal, wherein the one or more paddles squeeze a bag valve mask (BVM) attached to the ventilator device;
generate one or more outputs associated with a condition of the ventilator device; and
display, on a display device, at least one of the one or more outputs.

13. The device of claim 12, wherein the processor is further configured to determine a threshold delay percent that is set by an operator to determine a percentage of time at which a pressure drop below a threshold is ignored.

14. The device of claim 13, wherein the processor is further configured to use a threshold pressure value for determining when a next ventilation cycle in an assist mode will be triggered.

15. The device of claim 13, wherein a pressure difference between a mechanical positive end-expiratory pressure (PEEP) pressure value and a measured pressure is greater than a threshold pressure value, the controller device causes a compression based on the threshold delay percent.

16. The device of claim 12, wherein the ventilator device is enclosed in a pelican case.

17. The device of claim 16, wherein the pelican case comprises an intravenous (IV) attachment or a pole attachment.

18. A method comprising:
determining, by a processor of a ventilator controller device, one or more adjustable parameters associated with controlling a ventilator device to supply air to a user, wherein the one or more adjustable parameters are determined based at least in part on breathing thresholds associated with the user;
evaluating the one or more adjustable parameters to generate a control signal;
causing one or more paddles to articulate based at least in part on the control signal, wherein the one or more paddles squeeze a bag valve mask (BVM) attached to the ventilator device;
generating one or more outputs associated with a condition of the ventilator device; and
displaying, on a display device, at least one of the one or more outputs.

19. The method of claim 18, further comprising setting a threshold delay percent by an operator to determine a percentage of time at which a pressure drop below a threshold is ignored.

20. The method of claim 18, further comprising using a threshold pressure value for determining when the next ventilation cycle in an assist mode will be triggered.
